(19) United States
(12) Patent Application Publication
(10) Pub. No.: US 2008/0172107 A1
(43) Pub. Date: Jul. 17, 2008

(54) STAND ALONE OSTEOGENIC STIMULUS DEVICE AND METHOD OF USING

(76) Inventors: William J. McGinnis, Cincinnati, OH (US); Scott A. Metrick, Glenview, IL (US)

Correspondence Address: LADAS & PARRY LLP, 224 SOUTH MICHIGAN AVENUE, SUITE 1600, CHICAGO, IL, 60604, US

(21) Appl. No.: 11/622,178

(22) Filed: Jan. 11, 2007

(51) Int. Cl. A61N 1/05

(52) U.S. Cl. 607/51

(57) ABSTRACT

This invention presents a standalone osteogenic stimulus device and a method of using the device. The standalone osteogenic stimulus device includes a housing in which a pair of electrodes and a power supply are attached to the housing. The pair of electrodes and the power supply are coupled together to provide a therapeutic electric signal across the electrode pair. The device may be mounted in any osseous structure such as a fractured bone, a cracked bone, a weakened bone, a decalcified bone, a diseased bone, and even in a void where a portion of bone in order to simulate the healing process of the osseous structure. The method includes the acts of charging, closing, mounting, obtaining, opening, and sterilizing.

### FIELD OF THE INVENTION

The present invention relates generally to medical devices and more particularly to an osteogenic stimulus device for use in stimulating, fusing and healing osseous structures and tissue in the presence of an applied therapeutic electrical signal.

### DESCRIPTION OF THE PRIOR ART

The utilization of electric phenomenon to aid in expediting the healing of bone fractures or bone defects in a patient is well known in the art and has been the subject of numerous publications. Accordingly, a wide variety of electrical medical devices is currently available on the commercial market and an even larger number of these types of devices are known in the art of electrical medical devices, for example, the method for aiding formation of bone forming material disclosed by Kraus in U.S. Pat. No. 3,783,880; the constant current power pack for bone healing and method of use disclosed by Brighton et al. in U.S. Pat. No. 3,842,841; the tissue growth control apparatus and method disclosed by Greatbatch in U.S. Pat. No. 4,313,438; the bone growth stimulator disclosed by Jeffcoat and Wickham in U.S. Pat. No. 4,333,469; the bone and tissue healing device including a special electrode assembly and method disclosed by Christensen in U.S. Pat. No. 4,461,300; the combined tissue/bone growth stimulator and external fixation device disclosed by Tepper and Bryant in U.S. Pat. No. 6,678,562; the method and device for treating osteoarthritis, cartilage disease, defects and injuries in the human knee disclosed by Brighton and Pollack in U.S. Pat. No. 7,022,506; and the combination electrical stimulating and infusion medical device and method disclosed by Vilims in U.S. patent Publ. No. 2006/0155343. On treating fractured, injured and diseased osseous structures, such devices have ranged in size and complexity from large, bulky systems feeding electrical pulses by conductors extending through the skin.

Complications, including the possibility of infection, arise in the use of stimulators which have conductors extending through the skin. On the other hand, in the use of implanted stimulators, difficulties arise in providing suitable, operable stimulators which are small in size and in passing sufficient energy and control information to the stimulators, without direct connection, to satisfactorily operate them without direct connection.

While all of the above-described devices fulfill their respective, particular objectives and requirements, the aforementioned patents do not describe an standalone osteogenic stimulus device having the interconnected components of a housing, a pair of electrodes and a power supply.

This combination of elements would specifically match the user's particular individual needs of making it possible to provide a convenient means for electrically stimulating the healing process of an osseous structure. The above-described patents make no provision for a standalone osteogenic stimulus device having the interconnected components of a housing, at least one pair of electrodes and a power supply so that a therapeutic electrical signal may be applied across the pair of electrodes to aid in stimulating the healing process of a diseased osseous structure.

Therefore, a need exists for a new and improved standalone osteogenic stimulus device having the interconnected components of a housing, at least one pair of electrodes and a power supply so that a therapeutic electrical signal may be applied across a portion of an osseous structure in order to stimulate healing of the osseous structure.

In this respect, the standalone osteogenic stimulus device according to the present invention substantially departs from the conventional concepts and designs of the prior art, and in doing so provides an apparatus primarily developed for the purpose of providing a convenient and useful means for applying a therapeutic electrical signal across a portion of an osseous structure in order to stimulate healing of the osseous structure.

### SUMMARY OF THE INVENTION

The present device and method of using, according to the principles of the present invention, overcomes a number of the shortcomings of the prior art by providing a standalone osteogenic stimulus device and a method of using the standalone osteogenic stimulus for use simulating a healing process in an osseous structure. The standalone osteogenic stimulus device includes a housing, a pair of electrodes and a power source. The method includes the acts of charging, closing, mounting, obtaining, opening, and sterilizing.

In view of the foregoing disadvantages inherent in the known type a standalone osteogenic stimulus devices and method for use now present in the prior art, the present invention provides an improved a standalone osteogenic stimulus device, which will be described subsequently in great detail, is to provide a new and improved a standalone osteogenic stimulus device which is not anticipated, rendered obvious, suggested, or even implied by the prior art, either alone or in any combination thereof.

To attain this, the present invention essentially comprises a standalone osteogenic stimulus device having the interconnected elements of a housing, a pair of electrodes and a power supply.

There has thus been outlined, rather broadly, the more important features of the invention in order that the detailed description thereof that follows may be better understood, and in order that the present contribution of the art may be better appreciated.

The invention may also include a number of optional elements, such as an ion probe and a control circuit.

Numerous objects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved standalone osteogenic stimulus device that has many of the advantages of the prior standalone osteogenic stimulus devices and while minimizing a number of their disadvantages.

It is another aspect of the present invention to provide a new and improved standalone osteogenic stimulus device that may be easily and efficiently manufactured and marketed.

An even further aspect of the present invention is to provide a new and improved standalone osteogenic stimulus device that has a low cost of manufacture with regard to both materials and labor, and which accordingly is then susceptible of low prices of sale to the consuming public, thereby making standalone osteogenic stimulus devices economically available to the buying public.

Still another aspect of the present invention is to provide an standalone osteogenic stimulus device that provides a therapeutic electrical signal across, on, in and around an osseous structure via electrode pairs so that the healing of the osseous structure may stimulated.

Lastly, it is an aspect of the present invention to provide a new and improved method of using the device for stimulating the healing process of an osseous structure by implementing the acts of: charging, closing, mounting, obtaining, opening, and sterilizing.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1A shows two standalone osteogenic stimulus devices constructed in accordance with the principles of the present invention mounted within spinal disc;

FIG. 1B shows a standalone osteogenic stimulus device mounted in a femur bone;

FIG. 1C shows a standalone osteogenic stimulus device mounted in a jaw bone;

FIG. 2 is a cross sectional view of a standalone osteogenic stimulus device of the present invention; and

FIG. 3 is a partial cross sectional view of a standalone osteogenic stimulus device of the present invention.

The same reference numerals refer to the same parts throughout the various figures.

### DETAILED DESCRIPTION OF THE PREFERRED EMBODIMENT

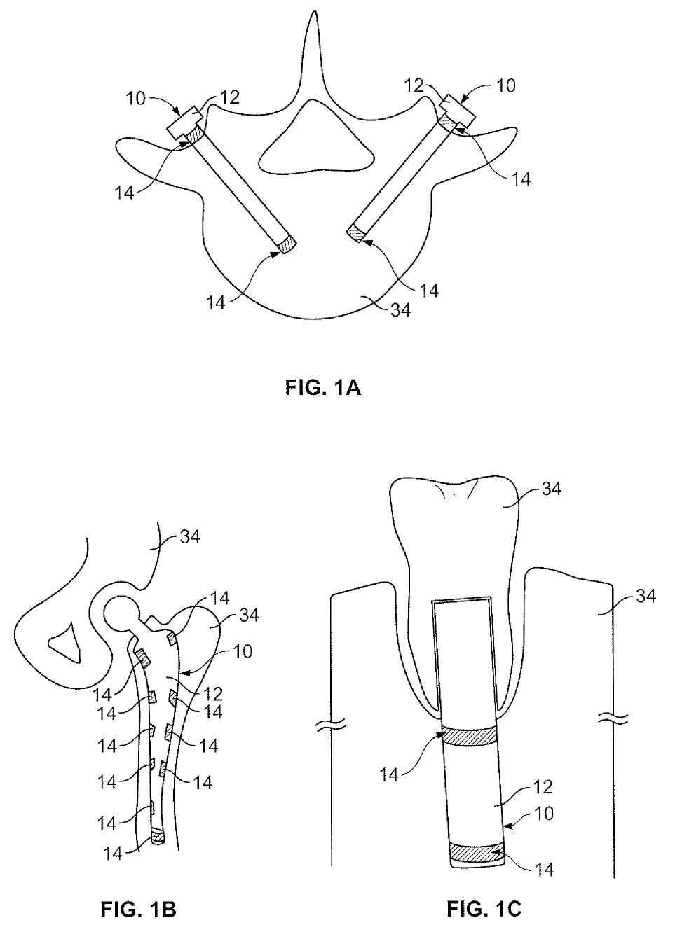

Referring now to the drawings, and in particular FIGS. 1 to 3 thereof, one preferred embodiment of the present invention is shown and generally designated by the reference numeral 10. One preferred embodiment of a standalone osteogenic stimulus device 10 comprises a housing 12, at least one pair of electrodes 14, and a power supply 16. Each pair of electrodes 14 attached to the housing 12. The power supply 16 is attached to the housing 12 in which the power supply 16 is coupled to the pair of electrodes 14 so that the power supply 16 and the pair of electrodes 14 are configured to apply a therapeutic electrical signal across the pair of electrodes 14.

The housing 12 of the standalone osteogenic stimulus device 10 may be any medically known standalone osteogenic housing 12 such as a pedicle screw, dental foundation plates, femur head prosthesis, bone bolt, bone plates, and dental implants. The housing may be mounted in any osseous structure such as a fractured bone 34, a cracked bone 34, a weakened bone 34, a decalcified bone 34, a diseased bone 34, and even a void where a portion of bone 34 once was.

An optional control circuit 18 may be added to standalone osteogenic stimulus device 10 in which the control circuit 18 is the attached to the housing 12. The optional control circuit 18 is coupled to the pair of electrodes 14 and coupled to the power supply 16 in which the control circuit 18 is configured to control the applied therapeutic electrical signal across the pair of electrodes 14. The control circuit 18 may comprise any number of various sub-circuitries such as a current limiter sub-circuit 20, a voltage regulator sub-circuit 22, a DC pulse sub-circuit 24, an impedance measurement sub-circuit 26, an application sub-circuit 28, and even an ion probe sub-circuit 32. The current limiter sub-circuit 20 can be configured to restrict the therapeutic electrical signal to a maximum of 20 milliamps across the pair of electrodes 14. The voltage regulator sub-circuit 22 can be configured to restrict the therapeutic electrical signal to a maximum of 2 volts across the pair of electrodes 14. The DC pulse sub-circuit 24 can be configured to cycle between a high and a low value of the therapeutic electrical signal across the pair of electrodes 14 in which the cycle period may be any duration, for instance having a cycle period of less than 1 minute. The high value of the voltage of the therapeutic electrical signal may be any magnitude such as 2 volts and the corresponding low value may be about 1 volt across the pair of electrodes 14. The high value of the current of the therapeutic electric signal may be any magnitude such as being about 20 milliamps and the corresponding low value may be about 5 milliamps across the pair of electrodes 14. The impedance measurement sub-circuit 26 may be coupled to the pair of electrodes 14 to the power supply 16 in which the impedance measurement circuit is configured to measure an electrical impedance across the pair of electrodes 14. The application sub-circuit 28 may be coupled to the impedance measurement sub-circuit 26 and to the power supply 16 in which the application sub-circuit 28 is configured to influence a change in a magnitude of the therapeutic application signal across the pair of electrodes 14 in response to the measured electrical impedance across the pair of electrodes 14.

The power supply 16 may be any commercially available power supply such as a conventional battery or a high capacity capacitor.

An optional ion probe 30 and an ion probe sub-circuit 32 may be added to the standalone osteogenic stimulus device 10 in which the ion probe 30 is attached to the housing 12. The ion probe sub-circuit 32 is coupled to the ion probe 30, to the power supply 16, and to the pair of electrodes 14, in which the ion probe sub-circuit 32 is configured to measure an ion signal from the ion probe 30 in which the ion signal is proportional to an ion accumulation phenomenon at an interface between the ion probe 30 and its surrounding. The ion probe sub-circuit 32 may be configured to influence a change in a magnitude of the therapeutic application signal across the pair of electrodes 14 in response to the ion signal from the ion probe 30. The ion probe 30 may be any commercially available ion probe 30 those selected from the group consisting of hydronium ion probe 30, a hydroxide ion probe 30, a calcium ion probe 30, a fluoride ion probe 30, a chloride ion probe 30, a potassium ion probe 30, and a phosphate ion probe 30.

Another preferred embodiment of the standalone osteogenic stimulus device 10 comprises a housing 12; at least one pair of electrodes 14; a power supply 16; and an ion probe 30. Each pair of electrodes 14 is attached to the housing 12 and a control circuit 18. The power supply 16 is attached to the housing 12 and coupled to the pair of electrodes 14 in which the power supply 16 and the pair of electrodes 14 are configured to apply a therapeutic electrical signal across the pair of electrodes 14. The ion probe 30 attached to the housing 12. The control circuit 18 is attached to the housing 12 and coupled to the pair of electrodes 14 and to the power supply 16 in which the control circuit 18 is configured to control the applied therapeutic electrical signal across the pair of electrodes 14. The control circuit 18 comprises a current limiter sub-circuit 20; a voltage regulator sub-circuit 22; an impedance measurement sub-circuit 26; an application sub-circuit 28; and an ion probe sub-circuit 32. The impedance measurement sub-circuit 26 is coupled to the pair of electrodes 14 and to the power supply 16 in which the impedance measurement sub-circuit 26 is configured to measure an electrical impedance across the pair of electrodes 14. The application sub-circuit 28 is coupled to the impedance measurement sub-circuit 26 and to the power supply 16. The application sub-circuit 28 is configured to influence a change in a magnitude of the therapeutic application signal across the pair of electrodes 14 in response to the measured electrical impedance across the pair of electrodes 14. The ion probe sub-circuit 32 is coupled to the ion probe 30, to the power supply 16, and to the pair of electrodes 14. The ion probe sub-circuit 32 is configured to measure an ion signal from the ion probe 30 in which the ion signal is proportional to an ion accumulation phenomenon at an interface between the ion probe 30 and its surrounding. The ion probe sub-circuit 32 is configured to influence a change in the magnitude of the therapeutic application signal across the pair of electrodes 14 in response to the ion signal.

One preferred embodiment of a method of using an standalone osteogenic stimulus device 10 comprises the acts of: charging, closing, mounting, obtaining, opening, and sterilizing. The step comprises obtaining the standalone osteogenic stimulus device 10 comprising: a housing 12; at least one pair of electrodes 14 attached to the housing 12; a power supply 16 attached to the housing 12, wherein the power supply 16 is coupled to the pair of electrodes 14 in which the power supply 16 and the pair of electrodes 14 are configured to apply a therapeutic electrical signal across the pair of electrodes 14; an ion probe 30 attached to the housing 12; and a control circuit 18 attached to the housing 12, the control circuit 18 coupled to the pair of electrodes 14 and to the power supply 16 wherein the control circuit 18 is configured to control the applied therapeutic electrical signal across the pair of electrodes 14 wherein the control circuit 18 comprises: a current limiter sub-circuit 20; a voltage regulator sub-circuit 22; an impedance measurement sub-circuit 26 coupled to the pair of electrodes 14 to the power supply 16, wherein the impedance measurement sub-circuit 26 is configured to measure an electrical impedance across the pair of electrodes 14; and an application sub-circuit 28 coupled to the impedance measurement sub-circuit 26 and to the power supply 16, wherein the application sub-circuit 28 is configured to influence a change in a magnitude of the therapeutic application signal across the pair of electrodes 14 in response to the measured electrical impedance across the pair of electrodes 14; and an ion probe sub-circuit 32 coupled to the ion probe 30, to the power supply 16, and to the pair of electrodes 14, wherein the ion probe sub-circuit 32 is configured to measure an ion signal from the ion probe 30 in which the ion signal is proportional to an ion accumulation phenomenon at an interface between the ion probe 30 and its surrounding, and the ion probe sub-circuit 32 is configured to influence a change in the magnitude of the therapeutic application signal across the pair of electrodes 14 in response to the ion signal. The charging step comprises charging the power supply 16. The sterilizing step comprises sterilizing the device 10. The opening step comprises opening an access route to an osseous structure. The mounting step comprises mounting the device 10 within an osseous structure, wherein the power supply 16 of the device 10 is charged. The closing step comprises closing the access route, wherein the therapeutic application signal applied across the pair of electrodes 14 aids in stimulating healthy bond development.

Referring now to FIG. 1A which depicts two standalone osteogenic stimulus devices 10 mounted within spinal disc bone 34. Each of the standalone osteogenic stimulus devices 10 are shown having a housing 12 shaped as a pedicle screws and having a single pair of electrodes 14.

Referring now to FIG. 1B which depicts a standalone osteogenic stimulus device 10 mounted in a femur bone 34 acting as a partial component of a hip joint prosthesis in which plurality of pairs of electrodes 14 are shown attached to the housing 12.

Referring now to FIG. 1C which depicts a standalone osteogenic stimulus device mounted in a jaw bone 34 acting as a foundation for an artificial tooth bone 34. A single pair of electrodes 14 are shown attached to the housing 12.

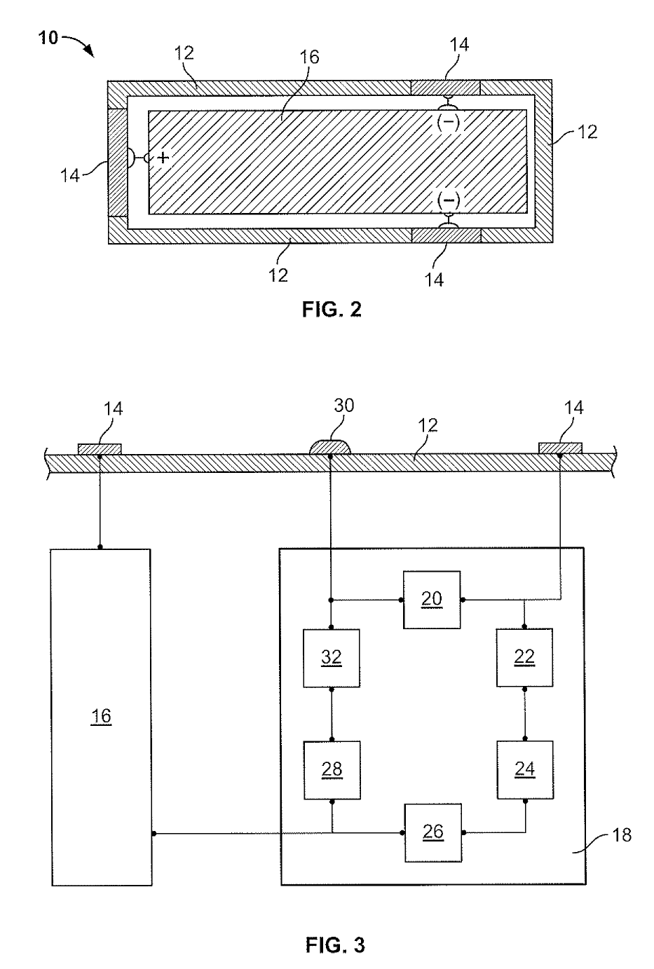

Referring now to FIG. 2 which depicts a cross sectional view of a standalone osteogenic stimulus device 10 having a housing 12 in which a pair of electrodes 14 and a power supply 16 are attached to the housing 12 of the device 10.

Referring now to FIG. 3 which depicts a partial cross sectional view of a standalone osteogenic stimulus device 10 showing the pair of electrodes 14 and the power supply 16 attached to the housing 12. Also shown is the optional ion probe 30 attached to the housing 12 of the device 10 and the control circuit 18 attached to the housing 12. The control circuit 18 is shown comprising a current limiter sub-circuit 20, a voltage regulator sub-circuit 22, a DC pulse sub-circuit 24, an impedance measurement sub-circuit 26, an application sub-circuit 28, and an ion probe sub-circuit 32.

As to the manner of usage and operation of the present invention, the same should be apparent from the above description. Accordingly, no further discussion relating to the manner of usage and operation will be provided.

While a preferred embodiment of the standalone osteogenic stimulus device 10 and associated methods for using the device 10 have been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification that fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A standalone osteogenic stimulus device comprising:
a housing;
at least one electrode pair attached to the housing; and
a power supply attached to the housing, wherein the power supply is coupled to the electrode pair in which the power supply and the electrode pair are configured to apply a therapeutic electrical signal across the electrode pair.

2. The device of claim 1 further comprising a control circuit attached to the housing, the control circuit coupled to the electrode pair and to the power supply wherein the control circuit is configured to control the applied therapeutic electrical signal across the electrode pair.

3. The device of claim 2 wherein the control circuit comprises a current limiter sub-circuit.

4. The device of claim 3 wherein the current limiter sub-circuit is configured to restrict the therapeutic electrical signal to a maximum of 20 milliamps across the electrode pair.

5. The device of claim 2 wherein the control circuit comprises a voltage regulator sub-circuit.

6. The device of claim 5 wherein the voltage regulator sub-circuit is configured to restrict the therapeutic electrical signal to a maximum of 2 volts across the electrode pair.

7. The device of claim 2 wherein the control circuit comprises a DC pulse sub-circuit configured to cycle between a high and a low value of the therapeutic electrical signal across the electrode pair.

8. The device of claim 7 wherein the DC pulse sub-circuit configured to have a cycle period of less than 1 minute.

9. The device of claim 8 wherein the high value is about 2 volts and the low value is about 1 volt across the electrode pair.

10. The device of claim 9 wherein the high value is about 20 milliamps and the low value is about 5 milliamps across the electrode pair.

11. The device of claim 1 wherein the power supply is selected from the group consisting of a battery power supply and a high capacity capacitor power supply.

12. The device of claim 2 wherein the control circuit comprises:
an impedance measurement sub-circuit coupled to the electrode pair to the power supply, wherein the impedance measurement circuit configured to measure an electrical impedance across the electrode pair; and
an application sub-circuit coupled to the impedance measurement sub-circuit and to the power supply, wherein the application sub-circuit configured to influence a change in a magnitude of the therapeutic application signal across the electrode pair in response to the measured electrical impedance across the electrode pair.

13. The device of claim 2 further comprising:
an ion probe attached to the housing; and
the control circuit comprises an ion probe sub-circuit coupled to the ion probe, to the power supply, and to the electrode pair, wherein the ion probe sub-circuit is configured to measure an ion signal from the ion probe in which the ion signal is proportional to an ion accumulation phenomenon at an interface between the ion probe and its surrounding, and the ion probe sub-circuit is configured to influence a change in a magnitude of the therapeutic application signal across the electrode pair in response to the ion signal.

14. The device of claim 13 wherein the ion probe is selected from the group consisting of hydronium ion probe, a hydroxide ion probe, a calcium ion probe, a fluoride ion probe, a chloride ion probe, a potassium ion probe, and a phosphate ion probe.

15. A standalone osteogenic stimulus device comprising:
a housing;
at least one electrode pair attached to the housing;
a power supply attached to the housing, wherein the power supply is coupled to the electrode pair in which the power supply and the electrode pair are configured to apply a therapeutic electrical signal across the electrode pair;
an ion probe attached to the housing; and
a control circuit attached to the housing, the control circuit coupled to the electrode pair and to the power supply wherein the control circuit is configured to control the applied therapeutic electrical signal across the electrode pair wherein the control circuit comprises
a current limiter sub-circuit;
a voltage regulator sub-circuit;
an impedance measurement sub-circuit coupled to the electrode pair and to the power supply, wherein the impedance measurement sub-circuit is configured to measure an electrical impedance across the electrode pair;
an application sub-circuit coupled to the impedance measurement sub-circuit and to the power supply, wherein the application sub-circuit is configured to influence a change in a magnitude of the therapeutic application signal across the electrode pair in response to the measured electrical impedance across the electrode pair; and
an ion probe sub-circuit coupled to the ion probe, to the power supply, and to the electrode pair, wherein the ion probe sub-circuit is configured to measure an ion signal from the ion probe in which the ion signal is proportional to an ion accumulation phenomenon at an interface between the ion probe and its surrounding, and the ion probe sub-circuit is configured to influence a change in the magnitude of the therapeutic application signal across the electrode pair in response to the ion signal.

16. The device of claim 15 further comprising a DC pulse sub-circuit configured to cycle between a high and a low value of the therapeutic electrical signal across the electrode pair.

17. The device of claim 15 wherein the current limiter is configured to restrict the therapeutic electrical signal to a maximum of 20 milliamps across the electrode pair.

18. The device of claim 15 wherein the voltage regulator is configured to restrict the therapeutic electrical signal to a maximum of 2 volts across the electrode pair.

19. The device of claim 15 wherein the power supply is selected from the group consisting of a battery power supply and a high capacity capacitor power supply.

20. A method of using a standalone osteogenic stimulus device, the method comprising the acts of:
obtaining the standalone osteogenic stimulus device comprising
a housing;
at least one electrode pair attached to the housing;
a power supply attached to the housing, wherein the power supply is coupled to the electrode pair in which the power supply and the electrode pair are configured to apply a therapeutic electrical signal across the electrode pair;
an ion probe attached to the housing; and
a control circuit attached to the housing, the control circuit coupled to the electrode pair and to the power supply wherein the control circuit is configured to control the applied therapeutic electrical signal across the electrode pair wherein the control circuit comprises
a current limiter sub-circuit;
a voltage regulator sub-circuit;
an impedance measurement sub-circuit coupled to the electrode pair and to the power supply, wherein the impedance measurement sub-circuit is configured to measure an electrical impedance across the electrode pair;
an application sub-circuit coupled to the impedance measurement sub-circuit and to the power supply, wherein the application sub-circuit is configured to influence a change in a magnitude of the therapeutic application signal across the electrode pair in response to the measured electrical impedance across the electrode pair; and
an ion probe sub-circuit coupled to the ion probe, to the power supply, and to the electrode pair, wherein the ion probe sub-circuit is configured to measure an ion signal from the ion probe in which the ion signal is proportional to an ion accumulation phenomenon at an interface between the ion probe and its surrounding, and the ion probe sub-circuit is configured to influence a change in the magnitude of the therapeutic application signal across the electrode pair in response to the ion signal;
charging the power supply;
sterilizing the device;
opening an access route to an osseous structure;
mounting the device within an osseous structure, wherein the power supply of the device is charged; and
closing the access route, wherein the therapeutic application signal applied across the electrode pair aids in stimulating healthy bond development.
